(19) United States
(12) Patent Application Publication
(10) Pub. No.: US 2004/0127898 A1
(43) Pub. Date: Jul. 1, 2004

(54) Bone fixing system

(76) Inventor: Michael Adam, Merlischachen (CH)

Correspondence Address: CHRISTIE, PARKER & HALE, LLP, 350 WEST COLORADO BOULEVARD, SUITE 500, PASADENA, CA, 91105, US

(21) Appl. No.: 10/682,745

(22) Filed: Oct. 9, 2003

(30) Foreign Application Priority Data
Nov. 4, 2002 (EP) 02 024 607.0
May 30, 2003 (EP) 03 012 456.4

(51) Int. Cl. A61B 17/56

(52) U.S. Cl. 606/64

(57) ABSTRACT

The invention relates to a bone fixing system, in particular for the repositioning of distal femoral fractures, comprising at least one nail, in particular a femoral medullary nail, and at least one screw, in particular a condyle screw, which can be led through a transverse bore formed in the nail, with a clamping device being provided which includes at least one spreading member which surrounds the screw at least region-wise, is axially adjustable relative to the screw and is spreadable at least in the region of the transverse bore during its displacement.

The invention relates to a bone fixing system comprising at least one nail and at least one screw which can be guided through a transverse bore formed in the nail.

Such systems in particular serve for the repositioning of distal femoral fractures such as are shown, for example, on page 141 in the “Manual der Osteosynthese” (Manual of Osteosynthesis), 3rd edition, Springer-Verlag, Authors: M. E. Müller, M. Allgöwer, R. Schneider, H. Willenegger. The nail is in particular a femoral medullary nail which is inserted from the side of the knee joint and which can be placed onto a target apparatus which allows the transverse bores formed in the nail to be located by a drill at the angle pre-determined by the target apparatus and corresponding to the orientation of the transverse bores and pre-bores to be made in the bone for the screws to be subsequently inserted. These screws, which are in particular provided in the form of condyle screws, serve to fix the medullary nail in the bone with respect to its axial direction.

Such a fixing system is also used when condyle fragments should be fixed with respect to the remaining bone by means of the screws inserted through the transverse bores of the nail. The medullary nail, which is held in the remaining bone and is fixed by means of locking screws there, in particular represents the only fixed reference basis for the fixing of the condyle fragments when the respective fracture is characterized by a plurality of condyle fragments. Examples for such fractures are shown in

FIGS. C1, C2 and C3 on page 141 in the aforesaid “Manual der Osteosynthese”.

It has been found to be problematic with such fractures that the bone screws serving for the fixing of the condyle fragments and guided through the transverse bores of the medullary nail are substantially freely movable with respect to their own axial direction relative to the medullary nail.

An implant is known from DE 197 23 339 C2 which includes a medullary space nail and a femoral neck nail and which serves for the fixing of fractures in the femoral neck region of the femoral bone. A clamping ring is provided to fix the femoral neck nail in a penetration opening of the medullary space nail, said clamping ring surrounding the femoral neck nail in the region of the penetration opening and being able to be expanded by means of a clamping element which can be displaced on the femoral neck nail in the axial direction.

It is the object of the invention to further develop a bone fixing system of the kind initially mentioned such that the nail can be fixed reliably and permanently in the most simple and secure manner possible, with it in particular being possible, in the repositioning of distal femoral fractures, to reliably fix a plurality of condyle fragments in their correct position with respect to the remaining bone.

This object is satisfied by the features of claim 1 and in particular in that a clamping device is provided which includes at least a spreading member surrounding the screw at least region-wise, being axially adjustable relative to the screw and spreadable at least in the region of the transverse bore during its displacement movement.

In accordance with the invention, the spreading member is axially displaceable and spreadable simultaneously at least in the region of the transverse bore. A particularly simply and reliably actuable clamping device can hereby be realized which provides a secure fixing of the screw in the bore of the nail.

In a particularly preferred embodiment of the invention, the spreading member can be spread by cooperation with at least one cross-sectional expansion of the screw, with it being the screw itself which automatically provides the spreading of the spreading member when this is moved axially relatively with respect to the screw during its displacement movement and cooperates with the cross-sectional expansion of the screw.

It is particularly preferred for the cross-sectional expansion of the screw to be provided in the form of a run-up ramp for the spreading member. A particularly uniform spreading of the spreading member can hereby be realized.

The fixing of the screw in the nail can be controlled in a particularly simple manner from the screw head if, in accordance with a further preferred embodiment of the invention, the spreading member extends over an axial length of the screw which extends from a region at the screw head, or in the proximity of the screw head, up to and into the region of the cross-sectional expansion of the screw. The spreading member is preferably of sleeve shape.

A particularly secure anchoring of the screw in the nail is achieved when, in accordance with a further embodiment of the invention, the spreading member has a plurality of clamping elements arranged distributed in the peripheral direction.

In a preferred variant of the invention, the spreading member has a plurality of clamping segments only separated from one another by elongate slots. Substantially the whole periphery of the spreading member, or least a substantial part of the periphery, is hereby available for the clamping of the screw in the nail. An exact alignment of the screw with the longitudinal axis of the transverse bore formed in the nail is hereby ensured.

In a further variant of the invention, the spreading member can have at least one pair of clamping tongues disposed diametrically opposite one another which preferably each cover an angular region which is smaller than the gap between the clamping tongues.

In a particularly preferred embodiment, just one pair of clamping tongues is provided, with the angular region covered by the clamping tongues preferably being so small that the spreading member is of a fork-like design. It has been found that such a comparatively slight covering of the periphery of the screw by the spreading member also allows a secure fixing of the screw in the transverse bore of the nail.

It is furthermore preferred for a cross-sectional expansion of the screw provided for the spreading member to lead from a reduced cross-section to the normal cross-section of the screw. The spreading of the spreading member thus takes place by the transition from the region with a reduced cross-section, in which the spreading member is arranged, into the region comprising the normal cross-section of the screw, with the normal cross-section being understood as the cross-section of a conventional screw which can be pushed through the transverse bore of the nail in an exact fit or with low radial clearance.

A weakening of the screw by cross-sectional reduction can be reduced to a minimum if, in accordance with a particularly preferred embodiment of the invention, the cross-section of the screw is exclusively reduced in regions provided for the clamping elements of the spreading member. The design of the spreading member already mentioned above is particularly advantageous in this case, according to which just one pair of clamping tongues are provided which together cover a comparatively small angular region and the spreading member is in particular of fork-like design, with the formation of groove-like or channel-like recesses or cut-outs, also termed key ways, in the screw for the clamping tongues being sufficient.

In accordance with a further preferred embodiment, the clamping device including the spreading member can be screwed to the screw. Provision is preferably made for this purpose for the screw to have a thread section in the head region for screwing to the clamping device.

It is particularly preferred for the clamping device to be made such that a screw movement of the clamping device can be converted into an axial actuation of the spreading member.

Whereas it is basically possible to make the clamping device in one part so that it only consists of the spreading member, with the spreading member simultaneously being axially displaced by being screwed to the screw, provision is made in accordance with a particularly preferred embodiment of the invention for the clamping device to be made in a plurality of parts and to include—in addition to the spreading member which can be pushed over the screw—a displacing member which can be screwed to the screw and which is or can be axially fixedly coupled to the spreading member. The coupling in particular takes place via a latch connection or snap connection.

It is preferred for the displacement member and the spreading member to be able to be rotated relative to one another in the axially fixedly coupled state. Only the displacement member of the clamping device has to be rotated hereby, whereas the spreading member carries out a purely translatory movement in the axial direction.

In a further particularly preferred embodiment of the invention, bounding means are provided which define a maximum penetration depth for the screw in the transverse bore of the nail. It is hereby made possible in a simple and reliable manner e.g. to draw a condyle fragment by means of the screw toward the nail and thus toward the surrounding remaining bone and, in so doing, to exert a more or less strongly pronounced compression action, depending on the application, before the screw is fixed in place by means of the clamping device in accordance with the invention in the transverse bore of the nail in the axial direction. Moreover, it is here exploited in an advantageous manner that the spreading member simultaneously acts as a rotational security for the screw.

Furthermore, provision can be made in accordance with the invention for the spreading member to be able to be spread apart simultaneously at a plurality of successive positions in the axial direction by cooperating with a profile of the screw.

It can be achieved by such a profile of the screw that the spreading member is driven radially outwardly over a comparatively large axial length when it is axially displaced relative to the screw. The length of this spreading distance can be directly pre-determined by the length of the profile formed at the screw. An advantage of this embodiment lies in the fact that a larger tolerance is present in the axial direction on the fixing of the screw in the nail. The total transverse bore of the nail can in this manner be used to fix the screw.

The spreading member can have a counter-profile at its internal side cooperating with the profile of the screw.

The profile of the screw and the counter-profile of the spreading member can be made complementary to one another.

Provision can furthermore be made for the profile of the screw and the counter-profile of the spreading member to each extend over an axial length which amounts to at least 50% of the length of the spreading member.

The profile of the screw can be formed by at least one screw section with a varying cross-section which repeats in the axial direction.

The profile of the screw can in particular include a plurality of inclined ramps arranged axially in succession.

In a possible embodiment, the profile of the screw is sawtooth-like.

In an alternative embodiment, the profile of the screw is wave-shaped.

The invention moreover relates to a screw, in particular to a condyle screw, for a bone fixing system such as has been explained above, with the screw being characterized in that it can be coupled to a clamping device which includes at least one spreading member which, in the coupled state, surrounds the screw at least region-wise and is axially displaceable relative to the screw, with the screw having at least one cross-sectional expansion by means of which the spreading member can be spread during its displacement movement.

The invention furthermore relates to a clamping device for a screw of a bone fixing system in accordance with the invention such as has been explained above, with the clamping device being characterized by at least one spreading member surrounding the screw at least region-wise, being axially displaceable relative to the screw and spreadable during its displacement movement by cooperation with the screw.

Further preferred embodiments of the invention are recited in the dependent claims, in the description and in the drawing.

The invention will be described in the following by way of example with reference to the drawing. There are shown:

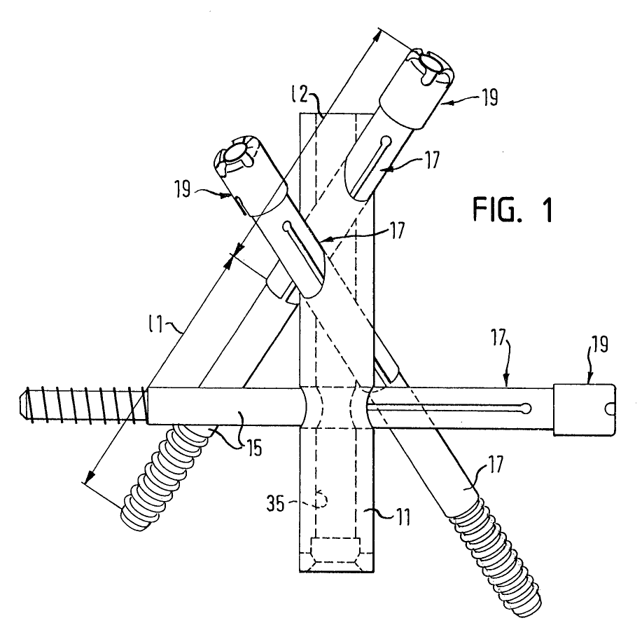

FIG. 1 a bone fixing system in accordance with a first embodiment of the invention;

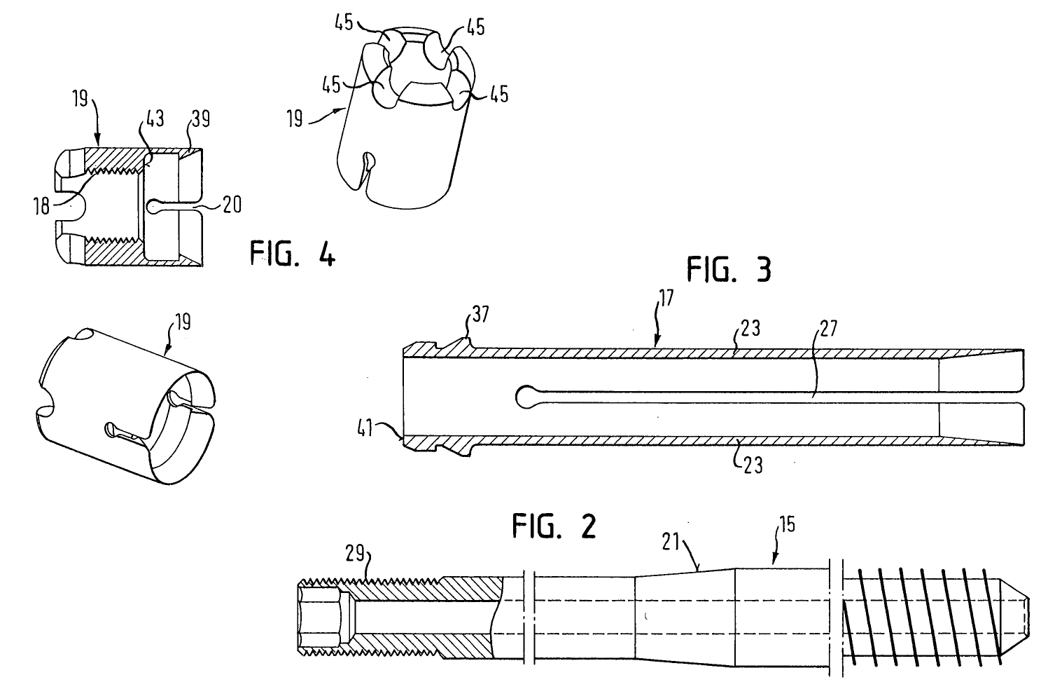

FIG. 2 a screw of the system of FIG. 1;

FIG. 3 a spreading member of the system of FIG. 1;

FIG. 4 various views of a displacement member of the system of FIG. 1;

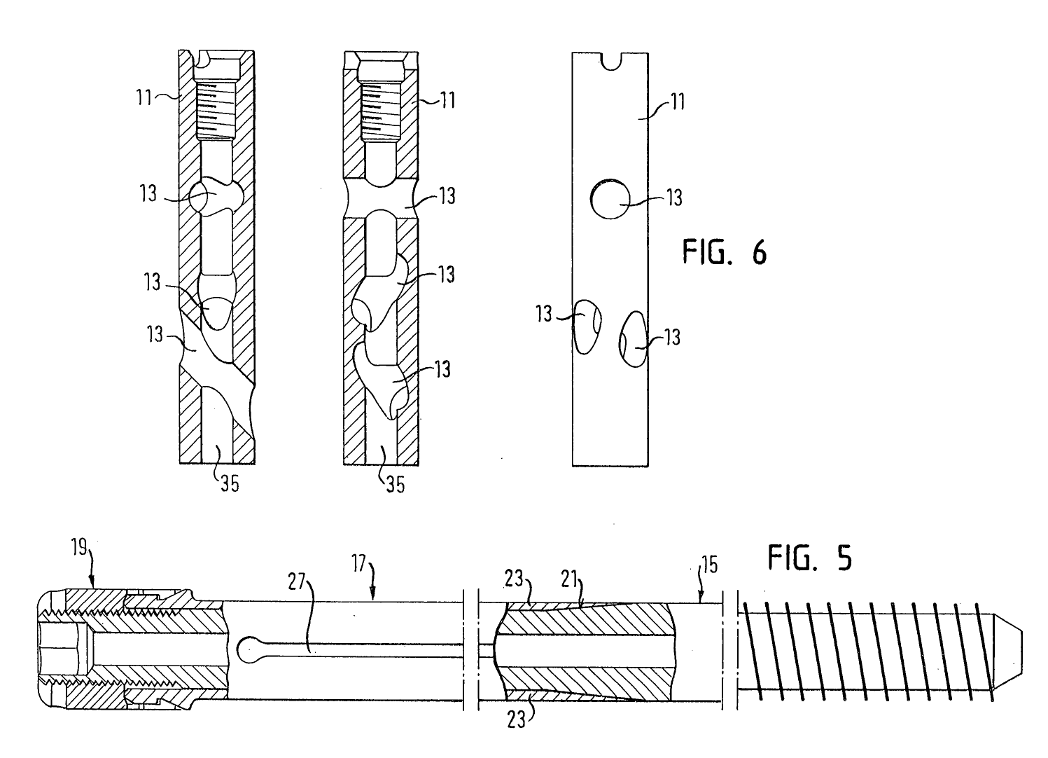

FIG. 5 a screw, a spreading member and a displacement member of the system of FIG. 1 in the mounted state;

FIG. 6 various views of a nail of the system of FIG. 1;

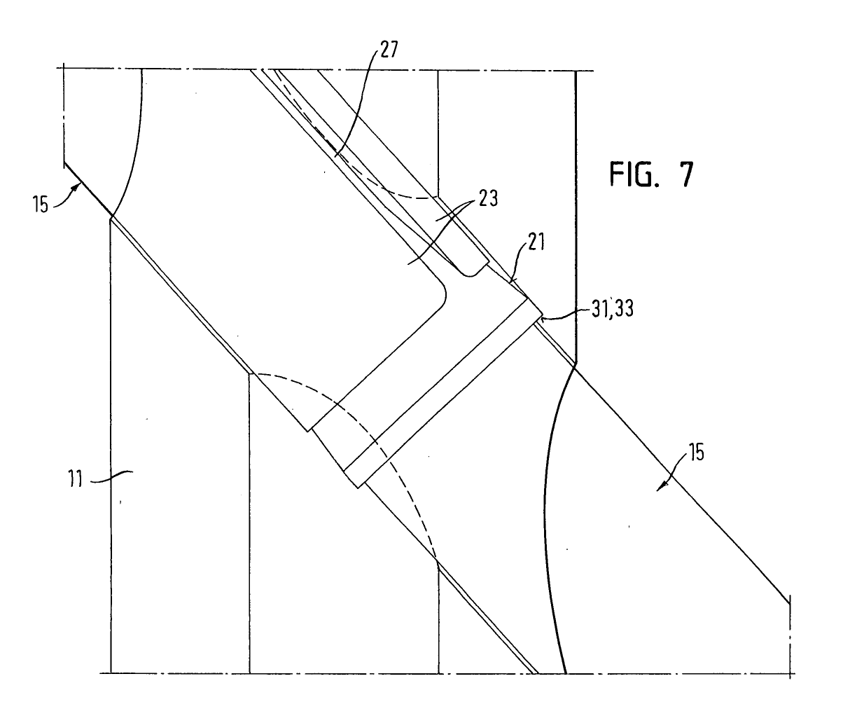

FIG. 7 in part, a bone fixing system in accordance with a second embodiment of the invention;

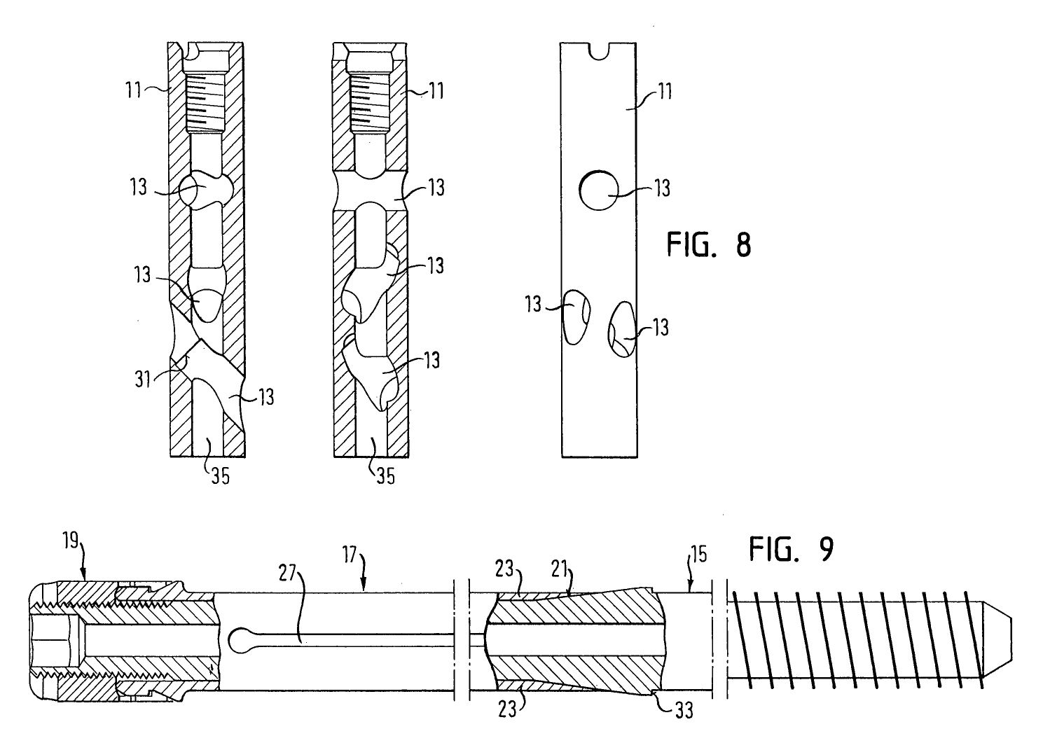

FIG. 8 various view of a nail of the system of FIG. 7;

FIG. 9. a screw, a spreading member and a displacement member of the system of FIG. 7 in the mounted state;

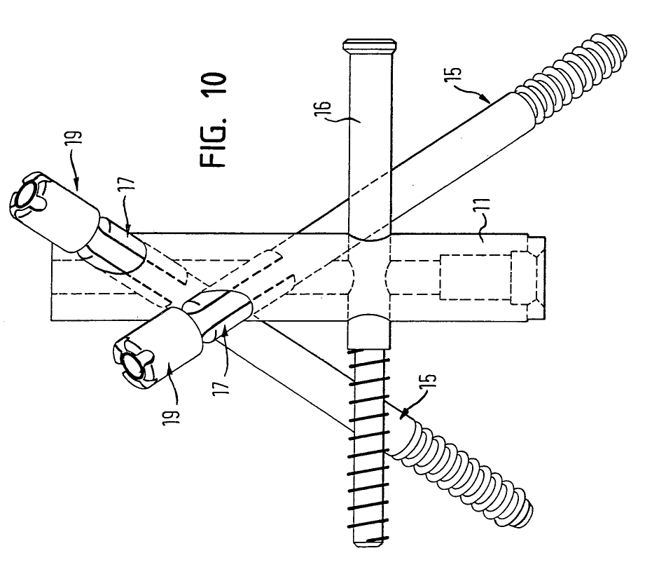

FIG. 10 a bone fixing system in accordance with a third embodiment of the invention;

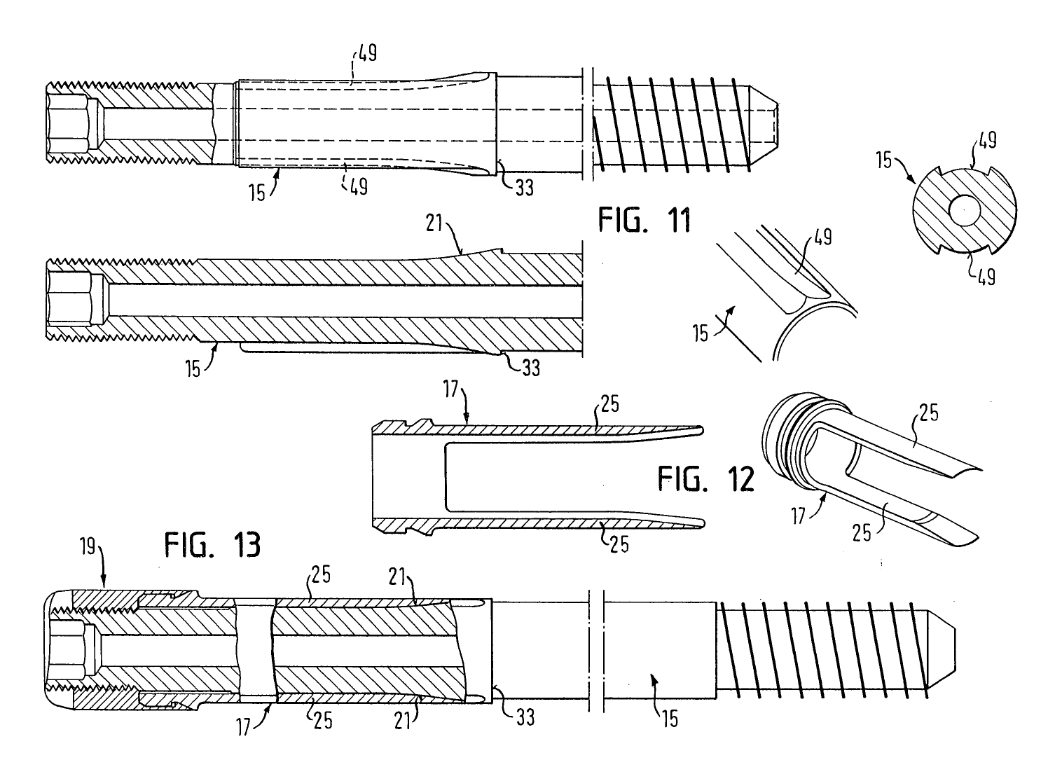

FIG. 11 various views of a screw of the system of FIG. 10;

FIG. 12 various views of a spreading member of the system of FIG. 10;

FIG. 13 a screw, a spreading member and a displacement member of the system of FIG. 10 in the mounted state;

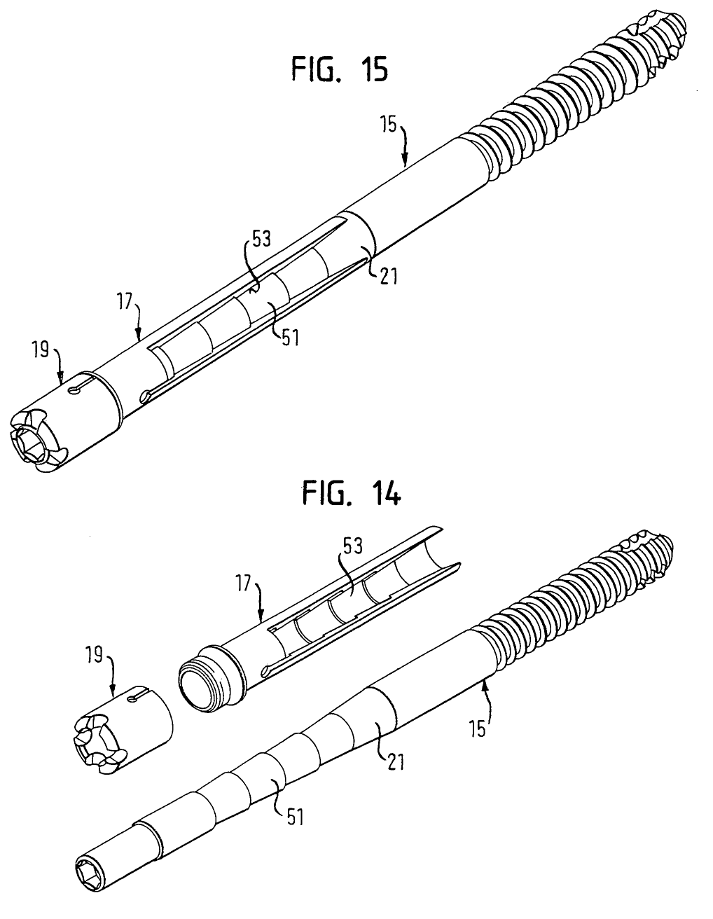

FIG. 14 a screw, a spreading member and a displacement member of a bone fixing system in accordance with a fourth embodiment of the invention;

FIG. 15 the parts of FIG. 14 in the assembled state;

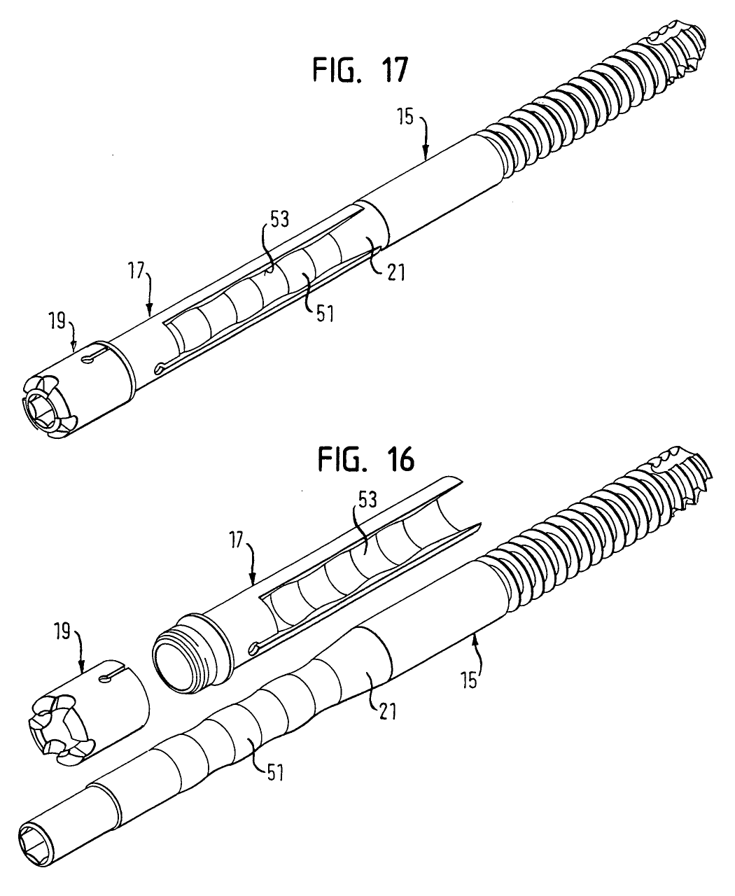

FIG. 16 a screw, a spreading member and a displacement member of a bone fixing system in accordance with a fifth embodiment of the invention; and

FIG. 17 the parts of FIG. 16 in the assembled state.

The bone fixing system in accordance with the first embodiment of the invention shown in FIGS. 1 to 6 includes, in the representation of FIG. 1, a femoral medullary nail 11 which is provided with a longitudinal bore 35 and three transverse bores, through which condyle screws 15 are inserted which are each fixed in the transverse bore of the nail 11 by means of a clamping device 17, 19 explained in more detail in the following and are thus fixed with respect to their axial direction.

Both the nail 1 and the screws 15 belong to a set of parts and are selected to match the respective application. Both the total length 11 and 12 of the screws 15 and the length 12 of the clamping device 17, 19, as well as the residual length 11 of the screw 15 can in particular adopt different values.

The screws 15 are each provided in the region of their free ends with a thread section which is only indicated in FIG. 1.

The condyle screws 15 of the invention in accordance with FIG. 2 in particular differ from conventional screws by an outer thread section 29 formed in the head region and a cross-sectional expansion provided in the form of a run-up ramp 21 in the embodiment shown. Seen from the head region in front of the ramp 21, the cross-section of the screw 15 is reduced over the total peripheral region such that the screw 15 has a conical shape in the region of the cross-sectional expansion 21. The run-up ramp 21 leads to the normal cross-section of a conventional condyle screw.

The spreading member 17 has a sleeve shape in accordance with FIG. 3 and has two elongate slots 27, which are disposed diametrically opposite one another, whereby the spreading sleeve 17 is divided into two clamping segments or flexural springs 23. With a constant outer diameter, the wall thickness of the spreading sleeve 17 reduces toward its free end in accordance with the cross-sectional increase of the screw 15 at the run-up ramp 21, whereby the free inner cross-sectional surface of the spreading sleeve 17 expands conically in the end region.

In the head region of the spreading sleeve 17, latch means 37 are formed via which the spreading sleeve 17 can be axially fixedly coupled to the displacement member 19 which is represented in FIG. 4, and is provided in the form of an adjustable screw and with corresponding latch means 39.

The coupling region of the adjustable screw 19 is provided with elongate slots 20 which, on the placing of the adjustable screw 19 onto the head region of the spreading sleeve 17, allow an elastic widening of the coupling region of the adjustable screw 19.

The adjustable screw 19 is furthermore provided with an inner thread section 18 via which the adjustable screw 19 can be screwed to the outer thread section 29 of the condyle screw 15.

The adjustable screw 19 furthermore has a central passage via which the head of the condyle screw 15 is accessible for a hexagon socket tool when the adjustable screw 19 is screwed on. The marginal region of the adjustable screw 19 at the front face is provided with four recesses 45 which are uniformly spaced apart from one another in the peripheral direction and at which a tool can be set for the turning of the adjustable screw 19.

FIG. 5 shows the mounted state with a still not spread sleeve 17. The cross-sectional reduction of the condyle screw 15 and the wall thickness of the spreading sleeve 17 are—including the transition region 21—matched to one another such that, with the exception of the head region, the arrangement of condyle screw 15 and pushed-on spreading sleeve 17 has a constant outer diameter.

The condyle screw 15 provided with the clamping device 17, 19 can be guided through one of the transverse bores 13 (cf. FIG. 6) formed in the nail 11. The penetration depth of the screw 15 in the transverse bore 13 of the nail 11 is defined by the axial position of the run-up ramp 21, i.e. the screw 15 is led so far through the transverse bore 13 until the run-up ramp 21, and thus the end region of the spreading sleeve 17, lie in the region of the transverse bore 13. By rotating the adjustable screw 19 relative to the condyle screw, the spreading sleeve 17 can then be moved in the axial direction, with (cf. FIGS. 3 and 4) a pressure surface 43 of the adjustable screw 19 loading the front face 41 of the spreading element 17.

Due to the rotationally symmetrical design of the adjustable screw 19 and of the spreading sleeve 17 in the coupling region, the spreading sleeve 17 does not rotate when the adjustable screw 19 is rotated, i.e. the screw movement of the adjustable screw 19 is converted into a purely axial translatory movement of the spreading sleeve 17.

During the axial adjusting movement of the spreading sleeve 17, the clamping segments 23 running up to the ramp 21 are pressed apart radially, whereby the sleeve 17 is spread, with the tapered free end of the spreading sleeve 17, which more or less runs to a tip, securely moving into the intermediate space between the condyle screw 15 and the inner wall bounding the respective transverse bore 13 of the nail 11, whereby a stable and permanent clamping of the condyle screw 15 in the transverse bore 13 of the nail is achieved and the condyle screw 15 is thus secured both against axial movements and against a rotation relative to the nail 11.

To release the clamping, the adjustable screw 19 is turned in the opposite direction, with a secure axial driving along of the spreading sleeve 17 being ensured by the latch or snap coupling between the adjustable screw 19 and the spreading screw 17.

The second embodiment of the invention shown in FIGS. 7 to 9 substantially differs from the first in that the condyle screw 15 is provided at the rear end region (seen from the screw head) of the run-up ramp 21 with a ring shoulder 33 which cooperates with a ring abutment 31 formed in the relevant transverse bore 13 of the nail 11 (cf. in particular FIG. 8) when the condyle screw 15 is inserted into the transverse bore 13.

The penetration depth of the condyle screw 15 in the transverse bore 13 is bounded by these abutment or bounding means 31, 33. Bone fragments can thus be drawn by means of the screw 15 toward the remaining bone into which the nail 11 is hammered.

Such abutment or bounding means 31 can be provided in each transverse bore 13 of the nail 11, with it also being possible, however, for one or more transverse bores 13 to have no such abutment for the condyle screw 15.

The third embodiment of the invention shown in FIGS. 10 to 13 in particular differs from the previously explained embodiments of the invention by the design of the spreading sleeve 17 and of the cross-sectional reduction of the condyle screw 15 provided for the spreading sleeve 17 in the region disposed in front of the run-up ramp 21.

The spreading sleeve 17 has only a pair of clamping tongues 25 which are disposed diametrically opposite one another and which each cover an angular region which is smaller than the gaps between the two clamping tongues 25 such that the spreading element 17 is in particular of approximately fork-like design (cf. in particular FIG. 12) overall.

The condyle screw 15 is provided—for the reception of the clamping tongues 25 of the spreading element 17—with channel-like or groove-like cut-outs 49 which are also termed key ways. In their end regions remote from the screw head, the depth of the key ways 49 gradually decreases, whereby the run-up ramps 21 for the clamping tongues 25 of the spreading element 17 are formed.

The weakness of the condyle screw 15 is reduced to a minimum by this cross-sectional reduction of the condyle screw 15 limited to the region of the clamping tongues 25.

The abutment or bounding means 33 explained in conjunction with the second embodiment of the invention in accordance with FIGS. 7 to 9 can also be provided in the condyle screws 15 of the third embodiment, as FIGS. 11 and 13 show. This is, however, not mandatory, i.e. the ring shoulder 33, on the one hand, and the key ways 49, on the other hand, are features of the condyle screws 15 in accordance with the invention which can generally be realized independently of one another.

FIG. 10 shows for the example of the screw 16 extending perpendicular to the nail 11 that all screws do not have to be provided with a clamping device in accordance with the invention in every application. It is rather the case—and this applies to all embodiments in accordance with the invention—that a “mixed” bone fixing system can also be used in which both conventional screws 16 and the clamping screws 15 of the invention are used.

The two embodiments shown in FIGS. 14 to 17 in particular differ from the previously explained embodiments in that the screw is provided with a surface profile 51 rotationally symmetrical with respect to the axis of the screw length in addition to the inclined ramp 21 cooperating with the free end regions of the spreading sleeve 17.

The profile 51 is disposed—considered in the direction of adjustment of the spreading sleeve 17—in front of the inclined ramp 21 and extends over a substantially axial length of the screw.

The inner side of the spreading sleeve 17 shown here in opened form simply for reasons of illustration, formed as a sleeve and having elongate slots is provided with a counter-profile 52 complementary to the profile 51 of the screw 15. When the spreading sleeve 17 is not spread open, the spreading sleeve 17 and the screw 15 combine to form a cylindrical whole with a constant cross-section in the axial direction which is equal to the screw cross-section subsequent to the inclined ramp 21.

If the spreading sleeve 17 is displaced in the axial direction by means of the displacement member 19 for the fixing of the screw in the transverse bore of a nail not shown here, the spreading sleeve 17 is not only spread open by the inclined ramp 21 in the region of its free ends, but also by the cooperation of the profile 51 of the screw 15 and of the counter-profile 53 of the spreading sleeve 17. A radial extension of the spreading sleeve 17 in the region between its free ends and its head region acted on by the adjustment member 19 therefore does not only take place due to the stiffness inherent in the spreading sleeve 17, but the spreading sleeve 17 is actively widened at a plurality of regions spaced apart in the axial direction by the profile 51 of the screw 15. Consequently, a clamping force can be exerted over a substantially axial region of the spreading sleeve 17, which makes it possible to utilize the total transverse bore of the medullary nail for the fixing of the screw 15.

In the embodiment of FIGS. 14 and 15, the profile 51 and the counter-profile 53 are sawtooth-like, whereas in the embodiment of FIGS. 16 and 17, a wave shape is provided for the profile 51 and the counter-profile 53. Each sawtooth or each wave peak of the screw profile 51 forms an elevation acting in the manner of an inclined ramp which forces a corresponding counter-section of the spreading sleeve profile 53 radially outwardly when the spreading sleeve 17 is axially displaced on the screw 15.

The sawtooth shape and the wave shape represent specific examples for an advantageous spreading apart of the spreading sleeve 17 over profiles and counter-profiles making possible a substantial axial region. Other profile shapes are quite possible, i.e. the not necessarily, but preferred, periodic axial course of the cross-section variation can deviate from the sawtooth and wave shape and generally include screw sections of any desired shape alternately forming—considered in the radial direction—elevations and depressions.

### Reference Numeral List

11 nail

13 transverse bore

15 screw

16 conventional screw

17 spreading member

18 inner thread section of the displacement member

19 displacement member, adjustable screw

20 elongate slot of the displacement member

21 cross-sectional expansion, run-up ramp

23 clamping segment

25 clamping tongue

27 elongate slot

29 thread section of the screw

31 ring abutment of the nail

33 ring shoulder of the screw

35 longitudinal bore of the nail

37 latch means of the spreading member

39 latch means of the displacement member

41 front face of the spreading member

43 pressure surface of the displacement member

45 recess

49 cut-out of the screw

51 profile

53 counter-profile

11 residual length of the screw

12 length of the clamping device

1. A bone fixing system, in particular for the reposition of distal femoral fractures, comprising at least one nail (11), in particular a femoral medullary nail, and at least one screw (15), in particular a condyle screw, which can be led through a transverse bore (13) formed in the nail (11),
characterized by a clamping device which includes at least one spreading member (17) which surrounds the screw (15) at least region-wise, is axially adjustable relative to the screw (15) and is spreadable at least in the region of the transverse bore (13) during its displacement.

2. A bone fixing system in accordance with claim 1, characterized in that the spreading member (17) is spreadable by cooperation with at least one cross-sectional expansion (21) of the screw (15).

3. A bone fixing system in accordance with claim 2, characterized in that the cross-sectional expansion (21) of the screw (15) is provided in the form of a run-up ramp for the spreading member (17).

4. A bone fixing system in accordance with claim 1, characterized in that the spreading member (17) extends over an axial length of the screw (15) which extends from a region at the screw head, or in the proximity of the screw head, up to and into the region of a cross-sectional expansion (21) of the screw (15).

5. A bone fixing system in accordance with claim 1, characterized in that the spreading member (17) is made in sleeve shape.

6. A bone fixing system in accordance with claim 1, characterized in that the spreading member (17) has a plurality of clamping elements (23, 25) arranged distributed in the peripheral direction.

7. A bone fixing system in accordance with claim 1, characterized in that the spreading element (17) has a plurality of clamping elements (23) only separated from one another by elongate slots (27).

8. A bone fixing system in accordance with claim 1, characterized in that the spreading member (17) has at least one pair of clamping tongues (25) which are disposed diametrically opposite one another and which preferably each cover an angular region which is smaller than the gaps between the clamping tongues (25).

9. A bone fixing system in accordance with claim 8, characterized in that just one pair of clamping tongues (25) is provided, with the angular region covered by the clamping tongues (25) preferably being so small that the spreading member (17) is of approximately fork-like design.

10. A bone fixing system in accordance with claim 1, characterized in that a cross-sectional expansion (21) of the screw (15) provided for the spreading member (17) leads from a reduced cross-section to the normal cross-section of the screw (15).

11. A bone fixing system in accordance with claim 1, characterized in that the cross-section of the screw (15) is only reduced in regions provided for clamping elements (23, 25) of the spreading member (17) and is provided for this purpose with groove-like or channel-like cut-outs (49).

12. A bone fixing system in accordance with claim 1, characterized in that the clamping device can be screwed to the screw (15).

13. A bone fixing system in accordance with claim 1, characterized in that the screw (15) has a thread section (29) in the head region for screwing to the clamping device.

14. A bone fixing system in accordance with claim 1, characterized in that a screw movement of the clamping device can be converted into an axial adjusting movement of the spreading member (17).

15. A bone fixing system in accordance with claim 1, characterized in that the clamping device is made in a plurality of parts and includes, in addition to the spreading member which can be pushed over the screw (15), a displacement member (19) which can be screwed to the screw (15) and is or can be axially fixedly coupled to the spreading member (17), in particular via a latch connection or snap connection.

16. A bone fixing system in accordance with claim 1, characterized in that the displacement member (19) and the spreading member (17) can be rotated relative to one another in the axially fixedly coupled state.

17. A bone fixing system in accordance with claim 1, characterized in that bounding means (31, 33) are provided which define a maximum penetration depth for the screw (15) in the bore (13) of the nail (11), with the bounding means preferably including a ring abutment (31) at the inner wall of the nail (11) bounding the bore (13) and a ring shoulder (33) formed at the screw (15).

18. A bone fixing system in accordance with claim 1, characterized in that the spreading member (17) can be spread apart at a plurality of successive positions in the axial direction simultaneously by cooperating with a profile (51) of the screw (15).

19. A bone fixing system in accordance with claim 18, characterized in that the spreading member (17) has a counter-profile (53) at its inner side cooperating with the profile (51) of the screw (15).

20. A bone fixing system in accordance with claim 19, characterized in that the profile (51) of the screw (15) and the counter-profile (53) of the spreading member (17) are made complementary to one another.

21. A bone fixing system in accordance with claim 19, characterized in that the profile (15) of the screw (51) and the counter-profile (53) of the spreading member (17) each extend over an axial length which amounts to at least 50% of the length of the spreading member (17).

22. A bone fixing system in accordance with claim 18, characterized in that the profile (51) of the screw (15) is formed by at least one screw section with a varying cross-section which repeats in the axial direction.

23. A bone fixing system in accordance with claim 18, characterized in that the profile (51) of the screw (15) includes a plurality of inclined ramps arranged axially in succession.

24. A bone fixing system in accordance with claim 18, characterized in that the profile (51) of the screw (15) is sawtooth-like.

25. A bone fixing system in accordance with claim 18, characterized in that the profile (51) of the screw (15) is wave-shaped.

26. A screw, in particular a condyle screw, for a bone fixing system, in particular in accordance with any one of the preceding claims, characterized in that the screw (15) can be coupled to a clamping device which includes at least one spreading member (17) which surrounds the screw (15) at least region-wise in the coupled stated and is axially displaceable relative to the screw (15), with the screw (15) having at least one cross-sectional expansion (21), by means of which the spreading member (17) is spreadable during its adjusting movement.

27. A screw in accordance with claim 26, characterized by the features of any one of claims 1 to 25 relating to a screw.

28. A clamping device for a screw of a bone fixing system, characterized by at least one spreading member (17) which is axially displaceable relative to the screw (15) and is spreadable during its adjusting movement by cooperation with the screw (1 5).

29. A clamping device in accordance with claim 28, when used with a bone fixing system, in particular for the reposition of distal femoral fractures, comprising at least one nail (11), in particular a femoral medullary nail, and at least one screw (15), in particular a condyle screw, which can be led through a transverse bore (13) formed in the nail (11),
characterized by the clamping device which includes at least one spreading member (17) which surrounds the screw (15) at least region-wise, is axially adjustable relative to the screw (15) and is spreadable at least in the region of the transverse bore (13) during its displacement.
